(19) United States
(12) Patent Application Publication
(10) Pub. No.: US 2013/0046265 A1
(43) Pub. Date: Feb. 21, 2013

(54) Disposable Underwear with Sanitary Pad

(71) Applicant: Kimberley R. Felix, N. Miami Beach, FL (US)

(72) Inventor: Kimberley R. Felix, N. Miami Beach, FL (US)

(21) Appl. No.: 13/210,692

(22) Filed: Aug. 16, 2011

(51) Int. Cl. A61F 13/496

(52) U.S. Cl. 604/385.01

(57) ABSTRACT

The disposable underwear with sanitary pads consists of underwear panties or briefs with a waistband and a cloth swathe. The cloth swathe has an inboard layer of liquid permeable material and an outer layer which is impervious to liquid. These items are sewn together along the waistband hem and the respective leg hole hems. Liquid absorbent material is disposed in the interspace or space between the inboard and outboard layers. Sometimes, the interspace is divided into patches and a crotch patch, midway between the waistband and the crotch from the posterior region to the anterior region, has a greater amount of absorbent material as compared with absorbent material in other patches.

The present invention relates to disposable underwear panties or briefs having liquid absorbent material as a sanitary pad. When the pad becomes partly or fully filled with bodily fluids, the panties or briefs are discarded and disposed of.

### BACKGROUND OF THE INVENTION

There is a need in the marketplace for disposable underwear which can be discarded when bodily fluids are released by a person. Women who menstruate may seek this type of disposable panties either in combination with other sanitary pads or tampons or during light flow days. Otherwise, adults with problems retaining bodily fluids may use these disposable panties which are thin, comfortable, and easy to wear.

### OBJECTS OF THE INVENTION

It is an object of the present invention to provide a disposable and discardable pair of panties or briefs.

It is another object of the present invention to provide underwear which has liquid absorbent material disposed between an inboard layer and a liquid impervious outer layer such that bodily fluids released by the person are captured in the absorbent material layer.

It is another object of the present invention to provide a method of personal hygiene which provides such uniquely designed panties or briefs enabling the captured bodily fluids and thereafter removing and discarding the panties or briefs with the released fluids.

### SUMMARY OF THE INVENTION

The disposable underwear with sanitary pads consists of underwear panties or briefs with a waistband and a cloth swathe which define two leg holes below the waistband. Hems define the leg holes and the waistband. The cloth swathe has an inboard layer of liquid permeable material and an outer layer which is impervious to liquid. These items are sewn together along the waistband hem and the respective leg hole hems. Liquid absorbent material is disposed in the interspace or space between the inboard and outboard layers. In a further enhancement, the interspace is divided into a plurality of patches and a crotch patch extends substantially midway between the waistband and the crotch from the posterior region to the anterior region of the user. In this crotch patch, a greater amount of absorbent material is disposed as compared with absorbent material in other patch interspaces. The method of personal hygiene provides the uniquely designed underwear panties or briefs, permits bodily fluids from the crotch to pass through the inboard layer thereby capturing the bodily fluids by absorption in the interspace between the inboard layer and the outboard layer. Fluid is blocked to the exterior of the panties by the outer layer. After removing the panties or briefs, the underwear is discarded and disposed of.

### BRIEF DESCRIPTION OF THE DRAWINGS

Further objects and advantages of the present invention can be found in the detailed description of the preferred embodiments when taken in conjunction with the accompanying drawings in which:

FIG. 1 diagrammatically illustrates an elevational anterior view of the disposable panties;

FIG. 2 diagrammatically illustrates a cross sectional view of an anterior panel or crotch panel from the perspective of section line A′-A″ in FIG. 1; and

FIG. 3 diagrammatically illustrates a further enhanced embodiment of the invention showing edge regions with smaller amounts of absorbent material as compared with a mid region.

### DETAILED DESCRIPTION OF THE PREFERRED EMBODIMENTS

The present invention relates to disposable underwear with an internally disposed sanitary pad. Similar numerals throughout the drawings designate similar items.

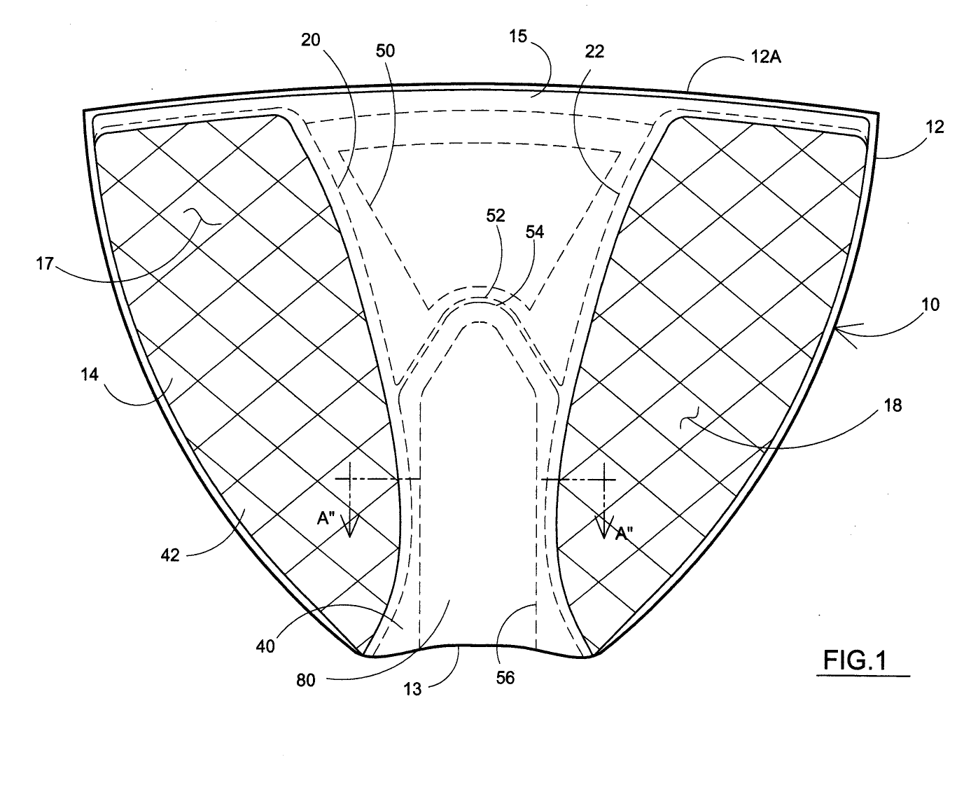

FIG. 1 diagrammatically illustrates disposable panties or briefs 10 having a waistband 12 with a waistband hem 12A. A cloth swathe 14 extends from the upper region 15 of waistband 12 over the anterior crotch portion of a person (not shown) leading to a posterior swathe portion and ultimately to the posterior of the waistband 12 (not shown). The cloth swathe 14 includes leg holes 17, 18 which are further defined by the cloth swathe with hems identified in the preferred embodiment as stitching 20, 22.

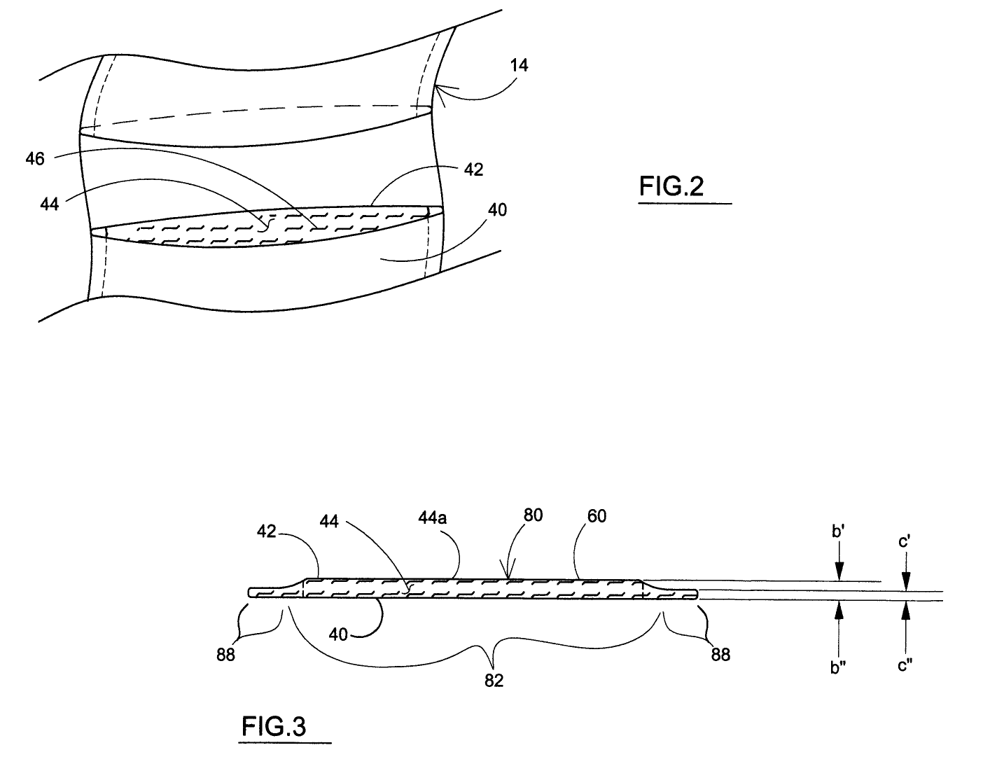

As best shown in FIG. 2, cloth swathe 14 includes an outer layer 40 which is imperious to liquid and an inner or inboard layer 42 which is permeable to bodily fluid and liquid. There is a space 44 between outer layer 40 and inner layer 42 called herein an “interspace” 44 which has liquid absorbent material 46 disposed throughout. In FIG. 1, inboard layer 42 is shown with large cross hatch square pattern to contrast it to outboard or outer layer 40.

FIG. 1 also shows a plurality of stitch lines 50, 52, 54 and 56. Stitch line 56 defines a crotch region spanning substantially vertically midway between waistband 12 and crotch 13 both in the anterior region of the panties of briefs as well as the posterior region. The crotch patch region 60 has substantially greater amount of absorbing material as compared with absorbing material in other patch interspaces such as the patch region defined by stitch 50.

FIG. 3 shows that the crotch patch 80 has a mid section 82 which has substantially more liquid absorbable material 44a as compared with end regions 86, 88. Material 44a is disposed in space 44. Further, the thickness b′-b″ of crotch patch 80 is almost twice as thick as the end regions 86, 88 as shown at lines c′-c″. In this manner, most of the bodily fluids released by the person are captured by the crotch patch as compared with other patches defined by sawing or other mechanism. The patches are defined by closure means defining compartments or chambers in space 44 between the outer layer an inner layer. This closure means could be stitching, adhesive or glue bond or seams, or thermal joints created by joining thermally the outer and inner layer together to form various patch regions. The method of personally hygiene provides this uniquely designed panties or briefs and permits the user to expel bodily fluids from the crotch. These fluids pass through the inboard layer and are captured by absorption in the interspace 44 between the inboard layer 42 and the outer layer 40. Fluid is blocked and cannot escape the panties or briefs by the liquid impervious outer layer. Thereafter, the user removes the panties or briefs after capture of the bodily fluids and discards them and disposes of the panties of briefs.

The types of women's panties include:
Types of Ladies Panty Underwear
hiphugger
high-leg brief
bikini
low rise bikini
string bikini
boyshort

The claims appended hereto are meant to cover modifications and changes within the scope and spirit of the present invention.

What is claimed is:

1. Disposable underwear with sanitary pad comprising:
underwear panties or briefs having a waist band and a cloth swathe defining two leg holes below the waist band;
a waist band hem and respective hems about said leg holes;
said clothe swathe having an inboard layer of liquid permeable material and having an outer layer of liquid impervious material, said inboard and outer layers sewn together at least at said waist band hem and said respective leg hole hems; and
liquid absorbent material disposed in the interspace between said inboard and outer layers.

2. A disposable underwear as claimed in claim 1 wherein said interspace is divided into a plurality of patches by closure means, said closure means including stitching, adhesive bonds and thermal joints, one patch covering a crotch region and extending to cover an anterior region substantially midway towards said waist band and extending to cover a posterior region substantially midway towards said waist band, the crotch patch having a patch interspace with substantially greater amounts of absorbent material as compared with absorbent material in patch interspaces over other areas.

3. A disposable underwear as claimed in claim 2 wherein said crotch patch interspace has substantially twice the amount of absorbent material as compared with absorbent material in patch interspaces over other areas.

4. A method of personal hygiene comprising:
providing underwear panties or briefs with a waist band bounded by a hem and a cloth swathe defining two leg holes below the waist band, each leg hole bounded by a respective leg hem; said clothe swathe having an inboard layer of liquid permeable material and having an outer layer of liquid impervious material, said inboard and outer layers sewn together at least at said waist band hem and said respective leg hole hems; and
permitting bodily fluids from a crotch to pass through said inboard layer;
capturing said bodily fluids by absorption in an interspace between said inboard and outer layers;
blocking fluid passage to the exterior of said panties or briefs with said outer layer; and
thereafter removing said panties or briefs after capture of said bodily fluids and discarding and disposing of said panties or briefs.
